(19) United States
(12) Patent Application Publication
(10) Pub. No.: US 2004/0072294 A1
(43) Pub. Date: Apr. 15, 2004

(54) Tissue reconstruction and regeneration

(76) Inventors: Susan J. Braunhut, Wellesley, MA (US); Kenneth A. Marx, Francestown, NH (US); Tiean Zhou, Lowell, MA (US); Anne Rugh, Dover Plains, NY (US)

Correspondence Address: FISH & RICHARDSON PC, 225 FRANKLIN ST, BOSTON, MA, 02110, US

(21) Appl. No.: 10/601,273

(22) Filed: Jun. 19, 2003

Related U.S. Application Data
(60) Provisional application No. 60/389,825, filed on Jun. 19, 2002.

(51) Int. Cl. C12P 21/02; C12N 5/06; C07K 14/475; A61K 38/24

(52) U.S. Cl. 435/69.1; 604/890.1; 435/325; 530/399

(57) ABSTRACT

The invention features improved methods of and apparatus for generating and administering treatment dosages of morphogens mobilized from extracellular matrices (ECM) for tissue reconstruction applications. In particular, the invention provides tissue reconstruction devices and methods that stimulate EDMs, e.g., by applying an electric potential, to achieve controlled release of morphogens that can then be placed into tissue sites when needed. Selectively releasing different morphogens at different times by the application of specific stimuli accelerates the tissue reconstruction process.

### RELATED APPLICATION

This application claims priority to U.S. Provisional Application Serial No. 60/389,825, filed on Jun. 19, 2002, the contents of which are incorporated herein by reference in its entirety.

### FIELD OF THE INVENTION

This invention relates to tissue reconstruction, regeneration, and repair. In particular, the methods and devices of the present invention accelerate tissue reconstruction.

### BACKGROUND OF THE INVENTION

Tissue reconstruction refers to the process in which, after injury, tissue regenerates and restores its function. Tissue reconstruction may also be desired when there is a defective tissue site, where there has been deterioration of tissue caused by an underlying disease that has lead to a loss of function. Tissue reconstruction is a complex process and involves cell migration, attachment, proliferation, and differentiation. For example, during wound healing, tissue reconstruction occurs in two distinct phases. First, in the inflammatory and stimulatory phase, blood cells and platelets aggregate to deposit granules, which promote fibrin deposition and stimulate the release of growth factors. Cells, e.g., leukocytes, are recruited from the circulation, proliferate and migrate to the wound site and begin to digest and transport debris away from the wound. Thereafter, in the second, non-proliferative or differentiation phase, granulation tissue forms and cells stop dividing and migrating. Fibroblasts, the key cell types in this phase, synthesize collagen to fill the wound and provide a strong matrix on which epithelial cells can attach and differentiate. As fibroblasts produce collagen, nutrients are supplied to the regenerating tissue and the migration of epithelial cells from the wound surface and stem cells from the surrounding tissue or bone marrow seals the wound, preventing the appearance of a scar and restoring the original tissue integrity to the site.

Morphogens, such as growth factors and differentiation factors, are important messengers in coordinating this complex orchestra of cellular events. Morphogens are a class of molecules with specificity for certain types of cells that can have either a proliferative or a differentiating effect depending upon the specific circumstances. Although the regulation and the precise mechanisms of action of morphogens have not yet been fully determined, the understanding of tissue reconstruction and the cascade of events necessary for the initiation of cell growth, migration, differentiation, and tissue regeneration, which collectively constitute the tissue reconstruction and regenerative process, has significantly increased.

### SUMMARY

The present invention features improved methods and devices for generating and administering treatment dosages of morphogens that are released from immobilized locations within portions of extracellular matricides (ECM) by stimulating the ECM, e.g., by an applied electric potential, a physical tension or strain, or other mechanical or chemical stimulus. These morphogens, once mobilized, are rendered soluble and can be applied to a tissue site, e.g., a wound or tissue defect, such as a diabetic ulceration, to accelerate the tissue reconstruction process. The invention also features tissue reconstruction devices and methods that use electric potential or pressure to initiate and control the release of morphogens into the site of tissue trauma. The invention further includes methods to produce morphogen compositions derived from ECMs, e.g., cell-free ECMs.

Selectively releasing different morphogens or groups of morphogens, such as growth factors and differentiation factor at different times by the application of electrical potential or physical tension to these ECMs at very small currents promotes, accelerates, and significantly improves the tissue reconstruction process.

In general, in one embodiment, the invention features methods of generating a morphogen composition from an extracellular matrix (ECM), by growing cells on a surface, e.g., a conducting surface, in a fluid under conditions and for a time sufficient to enable the cells to form an ECM; optionally removing cells from the ECM to form a cell-free ECM in a fluid; stimulating the extracellular matrix to release morphogens into the fluid; and collecting the fluid to form a morphogen composition.

In these methods, the morphogens can be growth factors, differentiating factors, bioactive fragments of the ECM, or any combination of two of more of these morphogens. In some embodiments, the morphogen composition includes a plurality of morphogens, and the fluid comprises a biocompatible liquid or biocompatible gel. In some embodiments, stimulating the extracellular matrix involves applying an electric potential to the extracellular matrix, e.g., an electric potential that cycles from a negative voltage to a positive voltage and back to a negative voltage. For example, the electric potential can range from −0.3 V to +0.3 V. The methods can also include varying the frequency, potential range, potential cycle shape, or potential cycle number of the electric potential to control release and activation of specific morphogens.

In another aspect, the invention features a morphogen composition that includes a plurality of morphogens released from a stimulated ECM, e.g., a cell-free ECM. The composition can further include a biocompatible fluid, such as a buffer or gel. The composition can be in lyophilized form, and the plurality of morphogens can include any two or more growth factors, differentiating factors, bioactive fragments of the ECM, or any combination of two or more of these morphogens, e.g., fibroblast growth factor, transforming growth factor beta, or both. The ECM can be stimulated by an electric potential, e.g., a positive, negative, or cyclic potential.

The invention also features bandages for application to a tissue defect such as a wound on the skin. The bandages include an impermeable membrane forming a sealed cavity; a first conducting layer arranged within the sealed cavity; a second conducting layer arranged within the sealed cavity and spaced apart from the first conducting layer; a buffer reservoir located within the sealed cavity; and an extracellular matrix, e.g., a cell-free ECM, arranged within the sealed cavity between the first and second conducting layers and contacting one of the conducting layers. They can further include a permeable membrane positioned adjacent to the cell-free extracellular matrix and arranged between the first and second conducting layers, and flexible insulating structural members to maintain separation between the first and second conducting layers during delivery of an electric potential to the cell-free extracellular matrix.

In these bandages, the impermeable membrane can include an upper impermeable membrane and a lower impermeable membrane sealed together at their respective edges to form the sealed cavity. In certain embodiments, the bandages are manufactured with an electrolytic buffer in the buffer reservoir. The lower impermeable membrane can be made to be removable.

In another aspect, the invention features an electric bandage for application to a tissue defect. This electric bandage includes a flexible sheet; a chamber fixed to the flexible sheet and containing an extracellular matrix, e.g., a cell-free ECM; a first conductor arranged on one side of the chamber; a second conductor arranged on another side of the chamber; an electric power source connected to the first and second conductors; a buffer reservoir arranged to deliver its contents to the extracellular matrix in the chamber; and a controller connected to the electric power source for applying an electrical potential to the extracellular matrix. In various embodiments, the buffer reservoir can be made of a liquid impermeable material, such as a polymer or plastic, that can be ruptured by pressure. The buffer reservoir can contain an electrolytic buffer. The ECMs in these electric bandages contain a plurality of morphogens that are bound within the ECM until released by application of electrical potential or pressure or other stimulus.

In specific embodiments, the first and second conductors can have a gold electrode surface, an indium tin oxide electrode surface, or an organic conducting polymer surface. For example, the organic conducting polymer surface can be electrochemically grown or deposited on a metal or non-metallic substrate. In general, the controller can apply an electric potential in a range of −0.3 V to +0.3 V.

In another aspect, the invention includes a pharmaceutical composition that includes a pharmaceutically acceptable carrier, such as saline, a buffer, a gel such as a hydrogel, and a morphogen composition.

The invention also features several methods. For example, in one aspect, the invention includes methods of tissue reconstruction by obtaining an extracellular matrix, e.g., a cell-free ECM; stimulating the extracellular matrix to induce release of morphogens; and administering the stimulated extracellular matrix to a site where tissue reconstruction is needed. These methods can further include incorporating the stimulated extracellular matrix into a bandage material. The sites to be treated can include tissue defects and the stimulated extracellular matrix can include specific morphogens for treating a specific type of tissue defect, e.g., a laceration, a burn, or a venomous sting. The bandages can be coded according to the specific type of tissue defect and morphogen that the extracellular matrix of the bandage has released.

Administering the stimulated extracellular matrix includes placing the bandage in contact with the tissue defect to saturate the tissue defect with morphogens, or placing the stimulated extracellular matrix in contact with a tissue defect in a surgical site to saturate the tissue defect with morphogens.

In other methods of tissue reconstruction, one obtains a morphogen composition as described herein and administers the morphogen composition to a site where tissue reconstruction is needed. The morphogen compositions can also be used for the manufacture of a medicament for use in tissue reconstruction by standard pharmaceutical formulating techniques.

As used herein, the term “releasate” refers to a mixture of a biocompatible fluid, such as a buffer or gel, e.g., a hydrogel, and substances released from a stimulated ECM, which contains one or more morphogens, such as transforming growth factor beta (TGF-β) and fibroblast growth factor (FGF).

The invention provides several advantages. For example, the new methods and devices allow the design of wound-specific or tissue-specific morphogen combinations for controlled release to an affected area of injury, trauma, or defect. The new wound healing devices and methods also store morphogens in a readily available reservoir and release them on demand using electric potential or other stimuli which are efficient and easy to harness. In addition, the new devices and methods accelerate wound healing and tissue reconstruction in the treatment of chronic as well as acute wounds, minimizing health care expenses and providing improved medical care and treatment. In general, quality of care is enhanced and the cost of health care decreases.

Furthermore, by generating and applying constant, known, and replicable treatment dosages of electric potential of defined specifications to the ECM, morphogens are released to treat particular types of wounds or defects. The electrical potential can be applied to bandage material containing the matrix. Once the current is turned off, the morphogens mobilized into solution by the current are applied with the bandage to the wound or defective area. The present device can be operationally efficient with low power requirements, in the range of less than three watts of average power (e.g., less than one watt). This allows the unit to be battery operated and small in size, thereby rendering the device portable and easy to use, as the small size facilitates transport and placement of the device, with minimal disruption, in a patient's treatment area during use.

Another benefit relates to the integration of multiple sensor and control circuits, which ensures precise treatment of different types of customized bandages enriched with matrices containing different complements of morphogens. In addition, these sensor and control circuits allow for unattended or layperson's operation of the device during treatment, providing for a highly simplified means of treatment. Moreover, because the dosages are predetermined for specific types of wounds or defects, the use of the present invention provides the correct treatment dosage for a variety of wounds or defects.

The present invention may be utilized in other treatment areas involving tissue reconstruction where increasing the rate of growth and proliferation of human or other living cells is essential, including the treatment of burns, venomous stings, and surgically implanted skin or soft tissue grafts, rehabilitation medicine, post surgical repair, and neuronal, brain, and spinal injury repair and regeneration. In addition to the medical treatment of soft tissue, the present invention has applications in the field of laboratory growth and manufacturing of skin grafts that are used in various surgical settings, veterinary medicine, and related clinical fields.

Additionally, the present invention provides significant benefits in treating military injuries. The accelerated wound healing process is critical in dangerous and hostile situations and the present invention will provide greater security, safety, and rapid treatment of a wide variety of wounds for military personnel.

Another benefit of the present invention is the discovery of new growth stimulators, growth inhibiting or differentiating factors, and other morphogens immobilized in the extracellular matrix (ECM) of cells. As a result, engineered synthetic or natural biological matrices containing bound growth stimulators, growth inhibitors, and differentiating factors can be regulated in their release using electrical potential or other stimuli. Additionally, the present invention involves the controlled release of morphogens that enables the morphogens to be stored prior to their release, while maintaining their potency and resisting degradation.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 is a schematic diagram of a Quartz Crystal Microbalance (QCM).

FIG. 2 is a flow diagram of a process of generating a cell-free extracellular matrix (ECM) used with a SMART BANDAGE™ of FIG. 7.

FIG. 3 is a schematic diagram of a cell culture vessel with a conductive material.

FIG. 4 is a schematic diagram of cell culture vessels with the conductive material of FIG. 3 with cellular material.

FIG. 5 is a schematic diagram of cell culture vessels with the conductive material of FIG. 4 with matrix material.

FIG. 6 is a schematic diagram of the conductive material of FIG. 5 ready to be incorporated into a bandage.

FIG. 7 is a cross-sectional view of a SMART BANDAGE.

FIG. 8 is a SMART BANDAGE device used to release morphogens prior to delivering the SMART BANDAGE of FIG. 7 to the wound site.

FIG. 9 is a cross-sectional, schematic view of a SMART BANDAGE that includes an integral power source.

FIG. 10 is a schematic diagram of the sizes of wound areas of mice treated with morphogens released by applying a specific electrical potential to a specific cell-free ECM or a control.

FIG. 11 is a cross-sectional view of an ECM mounted on a conductive support.

### DETAILED DESCRIPTION

Overall, the new tissue reconstruction devices and methods enable the mobilization of morphogens, e.g., growth stimulators, differentiating factors, and bioactive fragments of the ECM itself that are active in tissue reconstruction and repair, from natural or synthetic cell matrices by an applied stimulus, such as an applied electrical potential or an applied physical strain or tension. The applied stimulus is used to release morphogens stored in ECMs, e.g., cell-free ECMs.

First, morphogens, ECMs, and a Quartz Crystal Microbalance (QCM), which are used in a process of generating and monitoring the formation of a cell-free ECM are described, and in subsequent subsections, the details of individual steps and elements of the new tissue reconstruction devices and methods are presented.

Morphogens

Although the knowledge about the regulation of morphogens such as growth factors, differentiation factors, and bioactive fragments of the ECM itself, and their activities are not complete, expression of a given morphogen or its receptors can be induced by other morphogens and events, suggesting that sequences of morphogen-mediated messages networked across cell types and integrated with other signaling cascades are central to tissue reconstruction processes.

As a result, morphogens can serve as signals to regulate proliferation, migration, and interaction of cells critical for tissue reconstruction, such as that in wound healing and tissue regeneration in, e.g., diabetic ulcerations. For example, central to wound repair and defect reconstruction is the re-vascularization of the damaged tissue. Vascular endothelial growth factor (VEGF) is a growth factor that promotes proliferation and migration of endothelial cells. Stimulating expression of VEGF receptors in endothelial cell precursors induces endothelial cells to respond to VEGF secreted from other cells. Stimulating the release of VEGF from fibroblasts or other cell types (or stimulating VEGF production in endothelial cells) promotes migratory activity of endothelial cells. Also critical to tissue repair and regeneration is the establishment of the extracellular scaffold to support cell migration or proliferation.

In addition, stimulating release of agents such as fibroblast growth factors (FGF) from a number of cell types promotes proliferation and migration of fibroblasts, endothelial cells, smooth muscle cells, skin epithelial cells, which are all involved in the production of extracellular matrix materials such as collagen, laminin, and fibronectin. Further, stimulating FGF receptor production in fibroblasts capable of recognizing different types of FGF also plays a role in stimulating fibroblast activity and the production of extracellular matrix. Other morphogens implicated in tissue repair include insulin-like growth factors, and platelet derived growth factor, transforming, and epidermal growth factors. Morphogen molecules and their receptors are the likely molecular signals and regulators for tissue repair, and morphogen molecules also promote proliferation and migration of endothelial cells. Therefore, the activity of endothelial cells is critical to tissue repair and these cells represent the likely targets for morphogens and related molecules associated with the wound healing and regenerative processes.

Extracellular Matrices (ECMS)

Endothelial cells (EC) of blood vessels, i.e., capillaries, synthesize a basement membrane (BM) or extracellular matrix (ECM). The ECM can also be referred to as a biological scaffold. An epithelial cell remains viable within 1 mm of a capillary because of the diffusion distance of oxygen. Capillaries are the vessels that carry out oxygen exchange with the tissue because capillaries are only two cell diameters thick. Capillaries have a single endothelial cell on the lumenal side and a pericyte on the tissue side with an extracellular matrix sandwiched between the two cell types. The pericyte provides the strength and dilation capacity of the vessel wall and is aligned perpendicular to the flow. The endothelial cell carries out the exchange between tissue and blood and is aligned parallel to the flow. An extracellular matrix positioned between and synthesized by the two cell types is principally made of proteins including collagen IV, laminin, fibronectin, elastin, thrombospondin, and mucopolysaccarides, including heparin sulfate.

While in the ECM, morphogens such as FGF are sequestered and/or dormant. Once released, however, these growth and differentiation factors help to restore injured areas and rebuild damaged or defective tissue. During synthesis of the ECM, endothelial cells secrete and store within the ECM significant amounts of different morphogens. For instance, capillary endothelial cells of the cornea store 30% of the FGF in the underlying matrix that they produce, “Extracellular Matrix-Resident Basic Fibroblast Growth Factor: Implication For The Control of Angiogenesis,” Vlodavsky I., Fuks Z., Ishai-Mitchaeli R., Bashkin P., Levi E., Korner G., Bar-Shavi R., Klagsbrun M. J., 1991, Cell Biochem 45(2): 167-176. The capillaries contain the vascular cells responsible for new blood vessel growth when neovessels are needed following an injury or wound, and the stored growth stimulators, once released from the ECM, can rapidly contribute to tissue reconstruction such as wound healing.

Additionally, the release of FGF in combination with heparin makes the growth factor more active (e.g., 10 times more active). FGF, alone or in combination with heparin, stimulates ECs, SMCs (smooth muscle cells), pericytes, and fibroblasts, to proliferate and migrate. These cells can then restore or replace the cells needed to reconstruct the new tissue and vessels and restore the integrity of the wound.

However, FGF alone does not signal the cells to complete the reconstruction and the wound healing process. It takes the second phase of tissue reconstruction termed the differentiation phase to complete the restoration or closure of the wound. A second set of morphogens called differentiation factors are needed, to cause the cells to halt cell division and migration, and cooperate in re-constructing a well-integrated tissue compartment made up of connective tissue, vessels, and organs.

If the first growth phase is uninterrupted and the differentiation phase proceeds as expected, no scar tissue is formed and a complete tissue reconstruction results. Some tissues may carry out the growth phase or replacement of cells phase or in some cases, tissues accelerate into the differentiation stage before the first phase is complete, thereby disrupting cell replacement. In such cases, a scar is formed. The wound is eventually closed and in some cases, a scar, which has more tensile strength, is formed, thereby resisting and protecting against a second wound. One of the key morphogens involved in the second phase of tissue reconstruction is termed transforming growth factor beta (TGF-β). TGF-β is also found in storage in natural ECMs. Endothelial cells and pericytes of the capillaries produce TGF-α and secrete it readily. It then can become bound to the matrix that the two cells synthesize.

Other stimulatory growth factors besides FGF bind weakly or not at all to matrices, but are associated with tissue reconstruction and wound healing. One example is VEGF, as described in “Angiogenesis: An Update,” Diaz-Flores L., Gutierrez R., Varela, H. (1994), Histol Histopathol 9(4): 807-843. VEGF is produced primarily by hypoxic cells, i.e., cells which experience reduced oxygen supply, as often happens in wounds and injuries. VEGF is not constitutively made and thus new mRNA and translation of VEGF RNA precedes the appearance of VEGF at the wound site. VEGF has a signal peptide and is readily secreted by normal cells that are at risk of dying due to a lack of oxygen dissolved in blood. VEGF is more potent as a migration promoting or chemotactic factor for cells than as a proliferation inducer.

The ECM also provides storage for other types of morphogens, some of which are antagonists of FGF and VEGF action, such as transforming growth factor beta (TGF-β) mentioned above (Zhu H. J., Burgess A. W., “Regulation of transforming growth factor-beta signaling,” Mol Cell Biol Res Commun. 2001, 4:321-30; Derynck R., Akhurst R. J., Balmain A., “TGF-beta signaling in tumor suppression and cancer progression,” Nat Genet. 2001. 29:117-29; Ruscetti F. W., Bartelmez S. H., “Transforming growth factor beta, pleiotropic regulator of hematopoietic stem cells: potential physiological and clinical relevance,” Int J Hematol, 2001 74:18-25; Milani S., Calabro A., “Role of growth factors and their receptors in gastric ulcer healing,” Microsc Res Tech, 2001, 53(5):360-71.

TGF-β is produced in a latent state and is activated by proteolytic processing during tissue reconstruction, e.g., during the wound healing process. Once active, the TGF-β blocks the proliferative action of FGF and VEGF (“Transforming growth factor-beta signaling through the Smad pathway: role in extracellular matrix gene expression and regulation,” J Invest Dermatol, 2002, 118:211-5). The TGF-β is needed in the second phase of the wound healing process when cells are ceasing proliferation and are differentiating to form mature new tissue at the site of the wound. Thus, it appears that in normal ECM there is the simultaneous storage of growth promoting factors, e.g., FGF, and differentiating factors, e.g., TGF-β, which are natural antagonists of each other and are needed at distinct stages of the wound and defect healing process. It is also known that different types of healing will be facilitated or interfered with by the wrong morphogens being released. In normal lacerations, FGF and VEGF are required in the first phase and TGF-β in the phase to accomplish wound healing. In bums, only FGF and VEGF are desired, as the presence of TGF-β leads to significant scar tissue. In the absence of TGF-β, FGF and VEGF in combination with other factors will completely heal burns and results in no scar tissue. It is not known how cells control the timed or selective release of the factors they need as they proceed through the phases of tissue regeneration and healing. However, the current invention uses stimuli such as electrical potential to selectively release these factors from ECMs in a way that mimics the natural mechanism used by cells to regulate morphogen release.

Quartz Crystal Microbalance (QCM)

The quartz crystal microbalance (QCM) has been used to monitor thin film deposition in vacuum or gas. After it was shown that the QCM could be used in the liquid phase, the number of applications for the QCM has increased dramatically. A QCM consists of a thin quartz disc sandwiched between a pair of electrodes. Due to the piezoelectric properties of quartz, it is possible to excite the crystal to oscillation by applying an AC voltage across its electrodes deposited upon its upper and lower surface.

The resonant frequency (f) of the crystal depends on the total oscillating mass, including any mass coupled to the crystal. When a thin film is attached to the sensor crystal, the frequency decreases. If the film is thin, rigid, and dissipates no energy, the decrease in frequency is proportional to the mass of the film. In this way, the QCM operates as a very sensitive mass balance. Under these conditions, the mass of the adhering layer is linearly related to the attached mass. However, attached cells do not exhibit this linear behavior, but possess a more complex relationship reflected by the pattern frequency shifts measured as the cells attach.

Although solution-based QCM has been utilized mostly in the area of analytical chemistry, largely because of QCM's sensitivity and ability to measure liquid-solid interfacial phenomena, QCM can also be applied to biosensor systems involving biological macromolecules and living cells. Thus, for example, the QCM technique can be used to record the process of endothelial cell adhesion, spreading, and cellular mass distribution changes during initial cell to surface contact and homeostatic attachment.

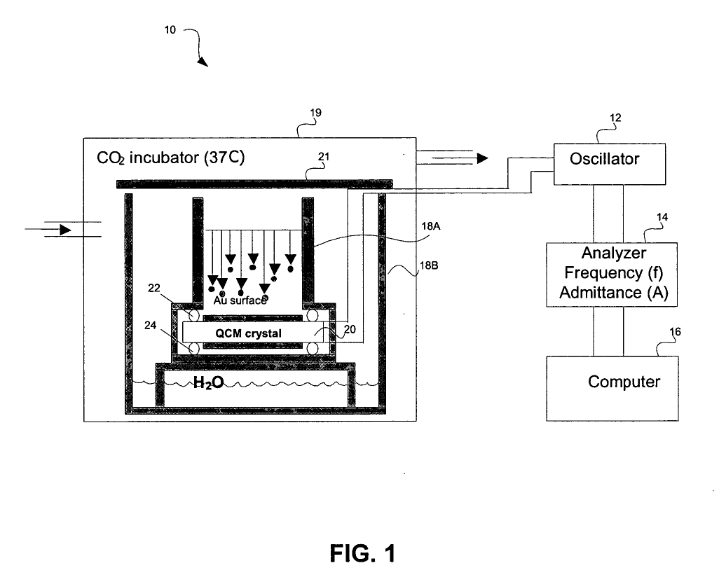

Referring to FIG. 1, an electrochemical QCM 10 is used as a cellular biosensor when endothelical cells are grown on its surface. The QCM 10 is an AT cut quartz crystal with a resonant frequency of 8.85 MHz with a gold upper and lower electrode (5 mm diameter). The QCM 10 includes an oscillator 12, a QCA 917 Quartz Crystal Analyzer 14 manufactured by SEIKO® EG&G CO., LTD., of Chiba, Japan, connected to a computer 16. The QCM 10 includes a cylindrical TEFLON® cell 18A that is placed in a water reservoir 18B in a CO₂ incubator 19, with a QCM crystal 20 that is sandwiched between two O-rings 22 and 24 to allow only the upper electrode to be exposed to the solution of interest. The TEFLON® cell 18A has a cover plate 21, and the CO₂ is maintained at 37° C.

Methodology

The new tissue reconstruction devices and methods induce angiogenesis, cell regeneration, and the growth of new blood vessels and tissue, by using natural or synthetic ECMs and engineering materials that store and then provide a controlled release of morphogens.

Using the new devices, one can stimulate ECM to release morphogens. For example, endothelial cells can be grown on a glass substrate coated with indium tin oxide to generate ECM. The cells and underlying ECM are then treated with a positive (e.g., +0.1, +0.2, +0.3, +0.4, or +0.5 V) or a negative (e.g., −0.1, −0.2, −0.3, −0.4, or −0.5 V) electrical potential for a sufficient time (e.g., 0.5, 0.7, 1.0, or 2.0 hours) and incubated with a medium for a sufficient time (e.g., 1, 2, 5, 10, 12, or 24 hours). The medium thus conditioned can then be tested for its effects on endothelial cell growth, e.g., as described below in Example 3. In general, the medium conditioned by the cell/ECM that has been treated with a positive electrical potential, e.g., +0.2 V, contains differentiation factors. In contrast, the medium conditioned by the same cell/ECM that has been treated with a negative electrical potential, e.g., −0.3 V, promotes cell proliferation. These results indicate that positive potential and negative potential selectively stimulate the release from cells and ECM of differentiating factors and stimulatory factors, respectively.

ECMs generated by pre-confluency and post-confluency endothelial cells provide different morphogens. Pre-confluency ECM and post- confluency ECM are treated with a negative electrical potential, e.g., −0.3 V, and used to condition media. The media can be tested for their activities on cell growth. The pre-confluency ECM-conditioned media generally promotes cell growth, while the post-confluency ECM-conditioned media generally inhibits cell growth. These results indicate that ECMs generated by cells at different stages store and/or release distinct morphogens.

The ability to selectively release different morphogens at different times using selective electrical potential at very small currents (e.g., nanoamperes) provides the required stimulus for the release of morphogens from isolated, morphogen-saturated natural and synthetic biological matrices for tissue reconstruction. This ability is exploited and used in forming the SMART BANDAGE™ device and other compositions and devices, described below.

For reconstructing tissue during wound healing, especially for treating chronic wounds, morphogens, e.g., growth factors and bioactive matrix molecule fragments, and other medicinal agents can be applied by the new devices described herein that use a stimulus such as electrical potential to release morphogens from their bound state in a storage matrix, that will in turn stimulate healing and reconstruction. These devices involve the use of applied electrical currents or pressure to liberate morphogens generated previously by cells and then stored in am ECM to stimulate growth, migration, and differentiation in the affected soft or solid tissues. The devices can utilize a discontinuous energy output to create pulsed electromagnetic energy or electric potential output of varying types to initiate (stimulate) or accelerate the mobilization of morphogens to speed the wound healing process.

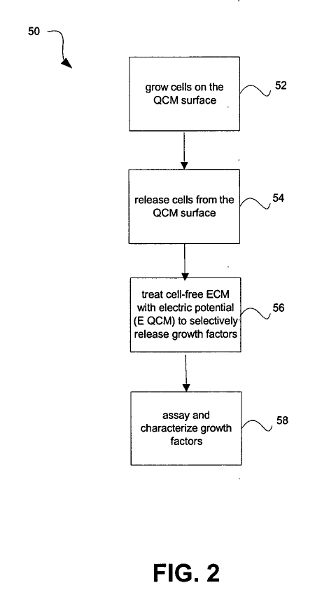

FIG. 2 illustrates a process for making the new tissue reconstruction devices and methods. A process 50 for generating a cell-free ECM begins by growing cells on the QCM surface. Cells, which form the ECM on any surface, can be removed from the ECM using a non-proteolytic and passive process which leaves behind the morphogen-enriched ECM on the surface. This can be done by treating the cells with EGTA or EDTA and washing detached cells with a phosphate buffered saline lacking calcium and magnesium. Accordingly, cells are released 54 from the QCM surface and the cell-free ECM is thereafter when needed treated with an electric potential to selectively release morphogens 56, which then can be specifically assayed and characterized 58. The matrix can be stored for at least a year prior to use and retain its morphogenic factors. The electrical potential can be applied using AC or DC current, as a continuous or pulsate energy, and varying the frequency and amplitude of the current used over a range, e.g., from about +0.3 to −0.3 V.

Specifically, in the body, the growth and differentiation effects that promote tissue reconstruction naturally are controlled by morphogens and their bioactive fragments released by the living and dying cells in an area undergoing tissue reconstruction. The new devices and methods described herein use morphogens and their bioactive fragments immobilized in isolated extracellular matrix material containing morphogens and their bioactive fragments previously made by cells rather than using the cells themselves. Cells store the morphogens, such as growth factors, they secrete in the ECM. In addition, when morphogens are bound to specific ECM molecules, the morphogens are more resistant to loss of their biological activity, and are thus more potent. For example, the same quantity of FGF can be ten times more stimulatory for the growth of endothelial cells when used in combination with molecules such as heparin sulfate, a glycosaminoglycan, which is present in several types of ECMs, including that made by endothelial cells.

Therefore, the new tissue reconstruction devices and methods are based on the binding affinities of morphogens and a stable period of dormancy of morphogens until they are needed. Cells store morphogens for long periods of time in the ECM to save critical time when emergencies arise that require the morphogens to be available in a short period of time. For example, in the case of wounds or oxygen deprivation, the rapid release of ready-made morphogens capable of stimulating wound healing or recruiting new oxygen bearing blood vessels into an area can prevent more extensive damage to the affected area of the wound.

As described herein, the new tissue reconstruction devices and methods utilize the enriched ECM produced by the cells, and from which the cells are subsequently removed intact and discarded. Additionally, cells can be applied to naturally occurring biological matrices or to synthetic matrices to deposit morphogens that the cells synthesize. The morphogens, in turn, become specifically bound and preserved until the growth factors are required.

Use of Stimulated ECM

As described above, electrical and other stimulation can be used to mobilize specific families of morphogen and their bioactive fragments in cell-free ECM, which can then be used to facilitate and accelerate the tissue reconstruction process. Thus, within the scope of this invention is an electrically stimulated cell-free ECM, and the liquid or gel collected from the electrically stimulated ECM, which is referred to herein as a releasate. The liquid or gel can be dried (e.g., by lyophilization) and stored, e.g., at about 4° C., in powder form. Before use, they can be reconstituted with a suitable solvent, e.g., saline or others.

Also within the scope of the invention is a pharmaceutical composition that contains a pharmaceutically acceptable carrier, diluent, or excipient, and a releasate prepared from the above-mentioned electrically stimulated cell-free ECM. The pharmaceutical composition can be used to facilitate tissue reconstruction. The pharmaceutically acceptable carrier, diluent, or excipient can include solvents, dispersion media, preservatives, humectants, chelating agents, antioxidants, stabilizers, emulsifying agents, suspending agents, gel-forming agents, ointment bases, penetration enhancers, or an antibacterial and/or antifungal agent. The releasate can be formulated into dosage forms for different administration routes utilizing conventional methods. For example, it can be formulated into lotion, solution, ointment, salve, cream, gel, hydrogel, dusting powder, pads, dressings, drenches, bandages, or plasters as generally known in the art for dermal or transdermal administration.

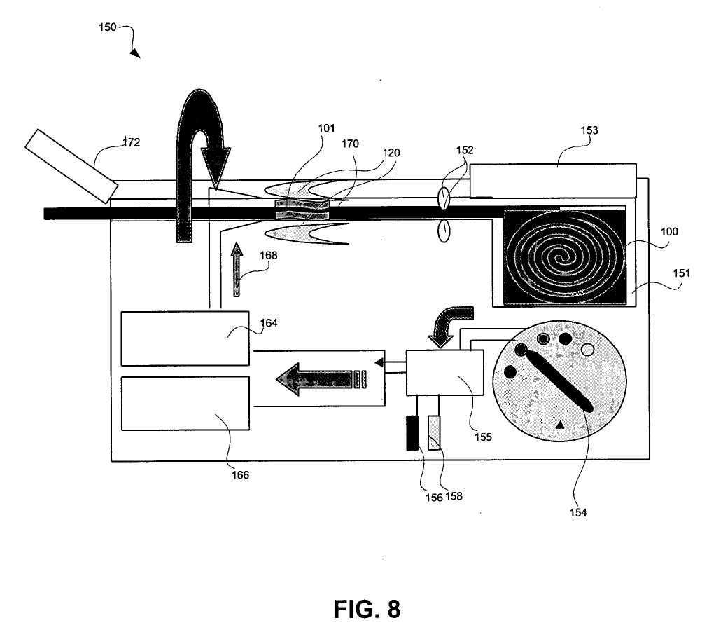

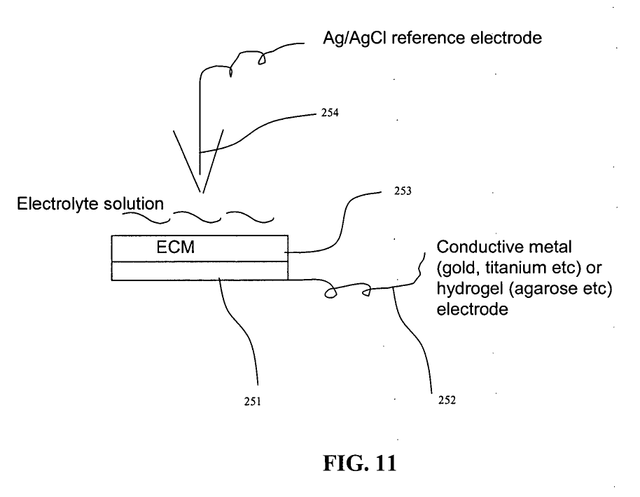

The above-described electrically stimulated ECM can be used directly in surgery. For this purpose, as shown in FIG. 11, a portion of an ECM 253 can be mounted on a support 251 such as, a disc, a string, a tape, a membrane, a micro-bead, or a particle (or used as is without a support if the ECM portion is sufficiently robust for handling). The ECM can be also mixed with or supported on a conductive polymer. To stimulate the ECM, one can contact the ECM-conductive polymer mixture with two electrodes (e.g., 254 and 252, similar to 120 in FIG. 8) and apply an electrical potential to the ECM in an electrolytic buffer. After being electrically stimulated, the ECM can be applied to a site of surgery to facilitate tissue reconstruction and wound healing. Such an ECM can be made on a biodegradable support, such as hydrogel. The support can be in various shapes for different applications. For example, one can use a tape-shaped support to repair a bone fracture by simply wrapping the support around the bone fracture. Similarly, one can stitch an incision after surgery with a thread-shaped support containing the electrically stimulated cell-free ECM. Since morphogens are released from the ECM, they promote bone repair and wound healing.

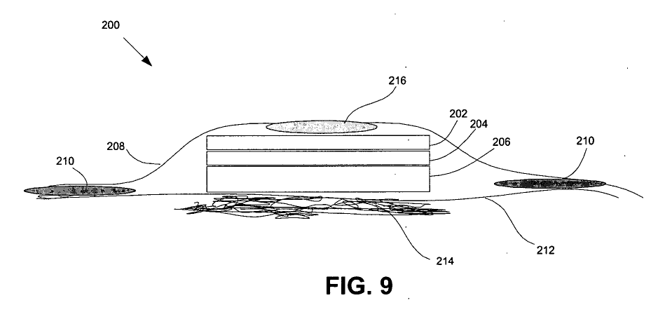

Another embodiment includes an intelligently designed bandage (i.e., a “SMART BANDAGE™”) tailored to facilitate tissue reconstruction in a specific tissue or to treat a specific type of wound. In general, the ECM material containing morphogens is incorporated into a wound dressing material (e.g., bandage) with a conducting device, such as polymer conductive surface, underneath the ECM, which remains preserved for a period of time. As required, a seal is broken above the matrix material to flood and saturate the ECM material with a buffer solution. Using a battery operated device, which can be located in a device that dispenses the bandage (FIG. 8) or incorporated into the bandage itself (see FIG. 9), the SMART BANDAGE is subjected to a brief electrical potential.

Different SMART BANDAGES are also provided for different types of wounds (e.g., burn wounds, lacerations, and venomous stings). Depending on the SMART BANDAGE type used, the bandage is enriched with a specific morphogen or bioactive molecule type deposited by a particular cell type, or a combination of morphogens are deposited by particular cell types. A battery-operated device may have a color-coded spin dial or push buttons to select the correct potential to be used for a particular wound type. Likewise, the SMART BANDAGE may be color-coded directly on the package material to distinguish ECMs enriched for growth factors best suitable for normal wounds, bums, venomous stings or bites (e.g., spider or bee venom). Each coded, e.g., color-coded, SMART BANDAGE has a different composite of ECM-bound morphogens produced by a different cell type and/or electrical potential specific to the type of wound. After receiving the specific potential needed, the SMART BANDAGE is placed in contact with a wound and the released morphogens move into the solution to saturate the wound or defect site.

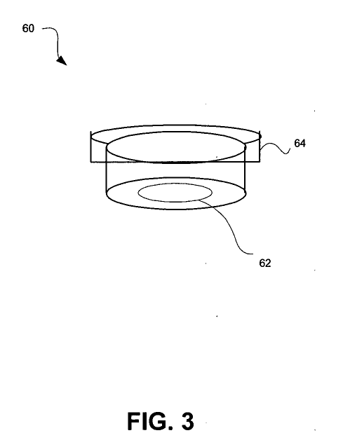

FIG. 3 illustrates one embodiment of a first step in producing the SMART BANDAGE. A thin flexible conducting material 62 for the SMART BANDAGE is placed into a cell-culturing dish 60, with a cover 64 to cover the growth area of the dish. The conducting material 62 can be made of metals or non-metal materials such as conductive polymers (e.g., polythiophene or polyaniline).

The conducting material 62 below the ECM may be modified in a variety of ways, such as applying biofilms, or by rendering metal surfaces more hydrophilic using chemical treatments, e.g., a treatment with 1:3 H₂0₂ (30%): H₂S0₄ at 80° C. These modifications can change the conducting material surface properties to encourage particular cell types to attach, grow, and affect the amount and type of ECM material synthesized by the cells on the conducting material. The properties of the surface provide for cell attachment and growth can alter the composition of the ECM and increase the ECM associated growth and differentiation factors. The surface properties can also be modified. For example, a biofilm may be added to the conducting surface. The use of a biofilm achieves more rapid cell attachment, spreading, and ECM synthesis, and a more uniform ECM coverage of the matrix surface, thus providing a more reproducible and effective tissue reconstruction process.

The modification procedure for adding a biofilm to the conducting material used in the SMART BANDAGE includes forming, at the conducting material surface, an electrochemically polymerized thin film of monomers that polymerize via radical polymerization following their redox alteration. This is accomplished prior to the plating of cells. Monomers including tyrosine and tyrosine derivatives can be utilized along with copolymerization of electrochemically short recognition peptides that have a terminal tyrosine, the polymerization site at the aromatic ring side chain structure. These short recognition peptides, covalently incorporated into the growing thin film, are used to match the recognition peptide sequences utilized by cells to bind to the underlying ECM. The specific short recognition site can also be short amino acid sequences that specifically recognize and bind some cell type's surface receptor. Different recognition peptides representing different extracellular matrix protein binding sites for these and other cells can be incorporated into the electropolymerized film in the same way.

Similarly, a whole range of other types of monomers can be electropolymerized via aromatic ring structures (e.g., pyrroles or thiophenes, anilines, phenols, and their derivatives). Moreover, any conducting surface, such as a metal (e.g., ITO or gold) or conducting polymer (e.g., polypyrrole or polythiophene, polyaniline) may be used. In addition to forming the film via electropolymerization, enzymatic polymerization using horseradish peroxidase or soybean peroxidase or other enzymes, also can be used to polymerize the same composite monomer films. The electrochemical synthesis approach or the enzymatic approach can be applied to modify a conducting surface to promote the attachment of cells via recognition peptides for use with the SMART BANDAGE. Forming a sufficiently thin film on a conducting surface allows for increased stimulation and morphogen release from the ECM on the surface of the biofilm.

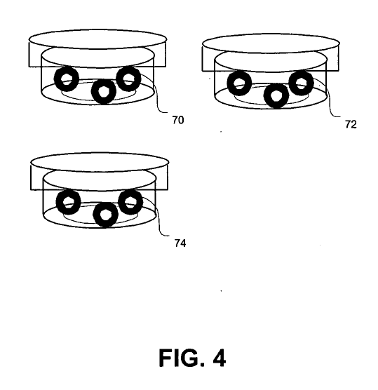

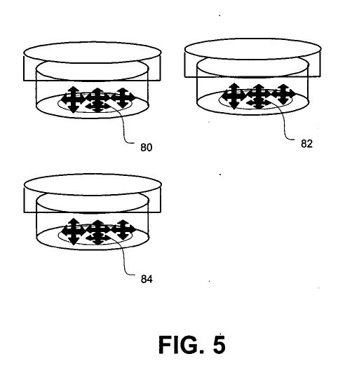

Next, as illustrated in FIG. 4, cells of varying types (70, 72, 74) are introduced into the cell culturing dishes 60 and allowed to grow and cover the conducting material 62 with or without the benefit of a biofilm. The cells 70, 72, 74 are selected on the basis of the morphogens they synthesize and incorporate into the ECM. Once the cells 70, 72, 74 have grown sufficiently to cover the conducting material 62 and had time to produce a morphogen-enriched matrix beneath them, the cells 70, 72, 74 are removed intact using calcium and magnesium chelating agents such as EGTA and EDTA. The cells 70, 72, 74 are rinsed with buffers that are calcium and magnesium free, then incubated with EGTA and or EDTA. Every two hours, the EGTA and/or EDTA is removed and refreshed. This process removes all the cells 70, 72, 74 passively and non-proteolytically, leaving behind intact matrices 80, 82, 84, which contain morphogens, on the conducting surface (FIG. 5) with or without a biofilm.

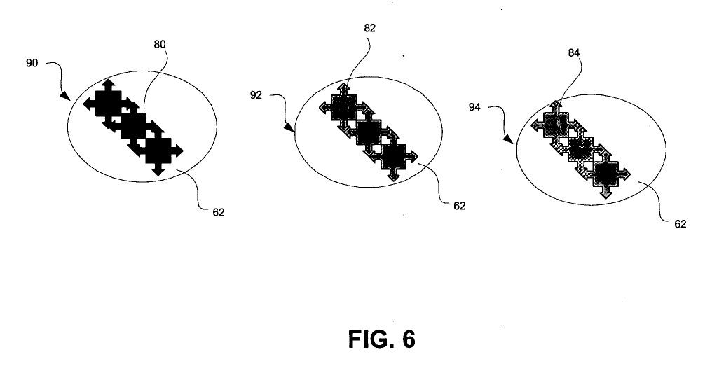

Thereafter, referring to FIG. 6, the conducting material 62 for the SMART BANDAGE is removed from the cell culturing dishes, and cut into the desired configuration to yield conducting material overlaid with ECM layers 90, 92, 94, that are incorporated into SMART BANDAGE devices.

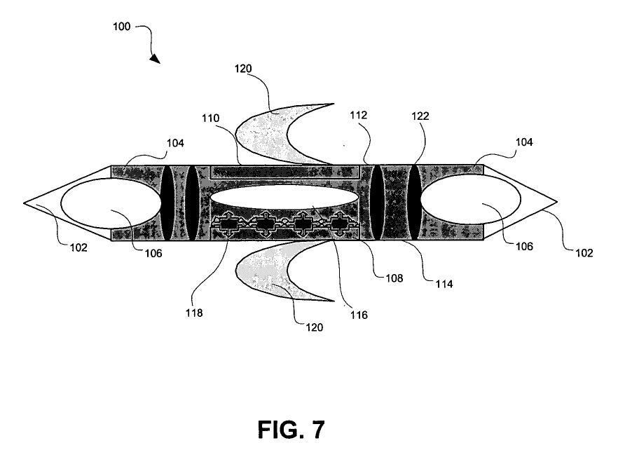

For example, using an ECM layer such as 90 in FIG. 6, a cross-section of a SMART BANDAGE 100 is shown in FIG. 7. The elements of the SMART BANDAGE 100 include water tight ends 102, an impermeable barrier to aqueous solutions 104, one or more buffer reservoirs 106, a lower conducting surface 108 (similar to 62 in FIG. 6), an upper conducting surface 110, an upper impermeable membrane 112, a lower impermeable membrane 114, a thin permeable membrane 116 (shown as a disk in FIG. 7) above the ECM 118, allowing electrical contact between the spring-mounted metal contact 120 and both the upper conducting surface 110 and lower conducting surface 108, above a conducting ECM layer 90, a spring-mounted metal contact 120 for the lower and upper conducting surfaces 108 and 110, and flexible structural struts 122 to maintain conducting surfaces separated during electric potential stimulation. The lower conducting surface 108 in combination with the ECM 118 (similar to 80 in FIG. 6) constitutes the ECM layer 90.

Referring to FIG. 8, the SMART BANDAGE 100 is loaded into a device 150. The device 150 is similar to an automatic film developer cassette and can be the size of a medium sized hand held flashlight or smaller. Depending on wound type, different types of the SMART BANDAGE 100 are threaded into position into a canister 151.

The canister 151 with lid 153 serves as a storage for the SMART BANDAGE 100, which can be loaded and unloaded like film in a camera. Upon demand, the SMART BANDAGE 100 is pulled through rollers 152 to crush the buffer reservoirs 106 (FIG. 7) releasing aqueous buffered electrolyte for potential stimulation onto the surface of the ECM 118. An ECM section 101 of the SMART BANDAGE 100 that includes the conducting ECM layer 90 is shown between the springs 120. A dial 154 is set by color codes, markers, words, letters, or some other designation code. Different codes represent different types of wounds. A code for a laceration, for instance, may have a red color-coded dial location, which when selected stimulates electrical potential on the ECM layer 90 that is enriched to treat lacerations. A green dial location (or push-button) may direct the bandage to treat bum wounds, and a yellow dial may direct the bandage to treat sting wounds. Electrical potential is applied for the appropriate time, e.g., several minutes, and the SMART BANDAGE is removed from device 150, the lower impermeable membrane 114 is removed, at least in the vicinity of the ECM 118, and the bandage is applied to the wound.

The liberated morphogens now in the buffer flow out of the bandage, e.g., through the conducting layer (or that layer can be removed as well when the impermeable membrane is removed), and saturate the wound site. The bandage can optionally include adhesive portions (e.g., that are exposed when the impermeable membrane is removed), or can be held in place using sterile tape. This process of stimulating a bandage and applying it to a wound can be repeated as needed. The electrical potential stimulation is selected and applied automatically by a pre-programmed microprocessor 155 having an ON switch 156 and OFF switch 158.

The device 150 also includes springs 120 which serve as metal contacts for the electrodes, powered by batteries 164, 166. The electrical potential 168 is controlled relative to a reference electrode 170 contained within the SMART BANDAGE 100 in contact with the buffer released from the crushed reservoir 106 (but not in contact with either the upper or lower conducting surfaces 108 and 110, respectively) and controlled by the pre-programmed microprocessor 150, are provided, as well as an opening flap 172 on the device 150.

As described previously, distinct morphogens, such as growth stimulators and differentiating factors, and other bioactive molecules are deposited by cells within biological ECMs. The new wound healing devices and methods use the knowledge of factor release and use the controlled release of morphogens from the cell-free ECM reservoir as needed to accelerate and promote wound healing. Although many of the protein morphogens residing in the ECM have been identified and characterized, novel lipids, bioactive ECM peptides and carbohydrates have also been discovered (“Vascular Endothelial Growth Factor and Vascular Adjustments to Perturbations in Oxygen Homeostasis,” Dor Y, Porat R., Keshet E., 2000, Am J Physiol Cell Physiol. 280(6): 1367-74; and “Role of Hypoxia and Extracellular Matrix-Integrin Binding in the Modulation of Angiogenic Growth Factors Secretion by Retinal Pigmented Epithelial Cells,” Mousa S. A., Lorelli W., Campochiaro P. A., 2000, J Cell Biochem. 74(1): 135-43). Hence, a variety of new growth stimulators and differentiating factors exist in the ECM.

In addition, in different embodiments, an individual SMART BANDAGE may be self-sufficient and independent of an electrical device such as device 150 of FIG. 8. A SMART BANDAGE of this type would be used individually with an integrated battery that can be used to apply electrical potential directly on the ECM layer contained in the SMART BANDAGE.

Referring to FIG. 9, an individual SMART BANDAGE 200 is formed by integrally combining a battery 202, a buffer reservoir 204, and an ECM layer 206 within a chamber, and a power ON/OFF switch 216 for allowing the user to control the application of electric potential to be delivered to the ECM layer 206 without the need for a separate battery or device, such as the batteries 164, 166 shown in device 150 shown in FIG. 8. The SMART BANDAGE 200 includes a flexible sheet 208, e.g., of tape, fabric, or film having an adhesive layer 210 which attaches the tape 208 to skin surface 212 to position the ECM layer 206 over a wound area 214 on the skin surface 212 or the defective tissue site. As described previously, depending on the type of wound, the wound area 214 is covered by a specific type of SMART BANDAGE 200 having a wound-specific ECM layer 206 that can release specific morphogens for specific types of wounds (e.g., lacerations, bums, stings).

Once the user has positioned the bandage 200, the user applies downward pressure on switch 216, which initiates the appropriate electric potential (e.g., −0.3 V), and also ruptures the buffer reservoir to allow buffer in the reservoir to permeate the ECM layer 206. The electric potential induces morphogens stored in the ECM layer to flow into the buffer and into the wound 214 to enhance the healing process. Alternatively, pressing the button can apply vibrations, e.g., from a piezoelectric device, or a tension or pressure, e.g., from a solenoid, to stimulate the ECM to release the stored morphogens.

### EXAMPLES

The following examples are illustrative and are not meant to be limiting.

### Example 1

### Mobilization of Morphogens

Capillary endothelial cells were cultured on indium tin oxide (ITO) coated glass. After allowing time for the cells to attach (about 24 hours) and synthesize an extracellular matrix (3-4 days), the cells were subjected to varying levels of electrical potential for one hour (e.g., +0.1, +0.2, +0.3, +0.4, or +0.5 V or a negative, e.g., −0.1, −0.2, −0.3, −0.4, or −0.5 V). Over the next 6 to 12 days, the cell numbers on at least triplicate ITO glass samples representing untreated and treated cells were determined using an automated system. Ranges of current (micro amperes) of specific electrical potential were determined for which capillary endothelial cells remained alive. In addition, cells that had been treated with a positive electrical potential of 0.2 V were differentiated whereas cells that were treated with a negative electrical potential of −0.3 V were stimulated in their growth. These were the first studies that lead to the conclusion that one type of electrical potential stimulated the growth of the cells and a second distinct type of electrical potential differentiated the cell growth over the next 6 to 12 days.

Different electrical potential during the one-hour treatment mobilized or liberated from the cells or from their ECM, or from both, different families of morphogens that are active in determining the growth and differentiation phases of the cells over the next 6 to 12 days. Additionally, the applied electrical potential was shown to mobilize morphogens from the ECM by the following experimentation.

### Example 2

### Mobilizing a Differentiating Factor

Endothelial cells or cells from the human breast cancer cell line, MCF-7, were grown on gold-coated quartz crystals such as the QCM 10 of FIG. 1. The gold-coated crystals upon which these cells grew are integral to the QCM for measuring the attachment, spreading, and adhesion of a monolayer of cells. Once the cells covered the working surface and deposited an extracellular matrix on the gold surface, the intact cells were removed intact, using the EGTA/EDTA incubation method as described above, leaving behind a biologically intact, cell-free extracellular matrix. The QCM detected and quantified the amount of material remaining in the ECM.

Subsequently, a negative electrical potential of 0.3 V was applied to the gold surface for a period of one hour at 37° C., which caused morphogens to be released from the ECM into the media. The resultant morphogen compositions or “releasates,” i.e., media conditioned by either endothelial cells or MCF-7 cells, were then tested for the presence of biologically active factors. The endothelial cell ECM releasate was shown to contain a potent differentiation factor with characteristics of the well-known differentiating factor, TGF-β. The active molecule blocked proliferation and migration of naive endothelial cells and was neutralized using a specific antibody to TGF β. This factor is active in the phase two period of wound healing and is critical for the successful and rapid closure of wound sites.

This experiment was repeated using the MCF-7 tumor cell matrix releasate, and no differentiation factors (which tend to inhibit cell growth) were discovered, indicating that the differentiation factor released by −0.3 V was specific for matrices generated by normal (not cancerous) endothelial cells. Additionally, in a cyclic voltammetric experiment, the potential was varied between −0.3 V to +0.3 V and back to −0.3 V for 5 cycles for the endothelial cell matrix. The results showed the release of a potent growth inhibitor that again resembled the same differentiation factor, TGF-β.

### Example 3

### Proliferation Assay

The media conditioned by the ECM was collected after receiving a negative or a positive electrical potential at 37° C. The ability of the harvested substances to modulate proliferation of endothelial cells was tested. For a proliferation assay, capillary endothelial cells were plated at 10,000 per well in a 48 well dish. The cells were allowed to attach for 24 hours and were then washed by replacing 150 μl of media. Wells in triplicate also received either a 50 μl addition of media alone, or media conditioned by a cell-free matrix derived from endothelial cells after a one-hour treatment with −0.3 V of electrical potential and conditioned for 24 hours. In triplicate, a time zero cell count was determined at this time and cells were counted in triplicate wells 3 and 6 days later using electronic coulter counting. Cells were re-treated with control or experimental media on day 3 for the 6-day treatment groups.

The result of the proliferation assay for capillary endothelial cells indicated that endothelial cells treated with the conditioned media were statistically inhibited in growth when compared to the day 3 and day 6 cell counts for cells receiving control media. These results indicated that treating endothelial cell derived ECM with an electrical potential of −0.3V liberated a potent endothelial cell growth inhibitory substance, which was likely a differentiating factor such as TGF-β.

Moreover, if a cyclic potential between −0.3 V to +0.3 V and back to −0.3 V was applied, varying the numbers of times, similar results were found. The morphogens liberated from these matrices by negative electrical potential have properties that are characteristic of TGF-β, a potent differentiating factor that inhibits growth and migration of endothelial cells while stimulating the cells to synthesize proteins and an extensive ECM to complete wound closure and increase tensile strength.

### Example 4

### Differentiation

The differentiating growth factors derived from the negative electrical potential treated cell-free ECMs play an important role in blocking migration and proliferation during the first phase of wound healing. They also facilitate the differentiation of endothelial cells to form functional blood vessels and scar tissue, and to promote wound closure.

For example, to test the effect of the factor on the migration and proliferation process, an endothelial cell monolayer was produced in 35 mm dishes. Using a plastic pipette, a single scratch or denuding area was produced from one side of the dish to another. At time zero, a sharp edge of demarcation was observed. In control cultures, open denuded areas of this dimension would be filled in by proliferation and migration of cells, by day 9.

In another experiment, a cross type area of denuding was produced in endothelial cell monolayers in a 48 well format. This allowed the use of material derived from a single QCM experiment, adding it in triplicate to identically denuded monolayers in three different wells. On day 3, cells were fixed, stained with Coomassie blue, and photographed. The denuded areas required several fields to capture the whole open area and images were imported into ADOBE® PHOTOSHOP® and made into a composite. The areas of potential regrowth, which remained denuded in triplicate wells were then measured, averaged for the two treatment groups, and compared using a Minitab® software package, and those possessing a p value less than 0.05 were determined to be statistically significant. The remaining open area of the control wounds (166 mm²) was half that of the treated denuded areas (328 mm²), thus, the degree of proliferation and migration in the presence of the conditioned media was reduced relative to the control cell cultures treated in parallel with control media by day 3.

This result was consistent with the presence of the differentiating factor, TGF-β, which are active in the second phase of wound healing when cells adequately proliferate and migrate, the second differentiation phase of the wound healing process begins. Cells cease migrating and proliferating in order to form a functional tube or vessel and form tight junctions which will allow for the passage of blood into the lumen of the new vessel and blood flow and oxygen delivery is restored. The differentiation phase is associated with cessation of division and migration. Thus, cell differentiation activity is an indication that differentiating factors are being released and are accelerating the final wound healing process, that of matrix formation and tissue organization and vessel function.

### Example 5

### Wound Healing in Mouse

An in vivo wound assay was conducted using a laceration model as described in Paul et al., 1997, Int. J. Biochem. Cell Biol. 29:211-20 and Kirker et al., 2002, Biomaterials 23(17):3661-71. Briefly, adult male C57 black mice, 4-6 weeks of age (25-35 g) were anesthetized with an intraperitoneal injection of 2.5% avertin (15 μL/g) and shaved with an electric razor on both sides of the back and disinfected with 70% ethanol. An ellipse was drawn on the skin of the mid-dorsal region on each side of the vertebral columns. A full thickness wound was created by excision of the area with a dermatological skin punch device. Both the epidermal and dermal layers were removed to make a full thickness wound.

ECM releasate and a PBS control vehicle were applied to the two wounds of each mouse, respectively. The ECM releasate was released from an ECM produced by bovine capillary endothelial cells by applying a −0.3 mV electrical potential to the matrix for one hour as described above. Four mice received 4 units of the releasate on their left wounds and four mice received 4 units of the releasate on their right side wounds. One unit activity was defined as that amount of releasate that can inhibit by 50% the growth of the cells when compared to untreated parallel cultures of cells over a 3 to 6 day period as described in Example 3 above. The second wound of each mouse received an equal volume of the PBS control vehicle.

The wounds were photographed with a digital camera and long axis and short axis were measured using a digital vernier caliper at time zero, days 3, 7, 9, 11, and 14. The size of the area of each elliptical wound was then calculated using the following formula:

Elliptical area (“EA”)=(π×long axis×short axis)/4.

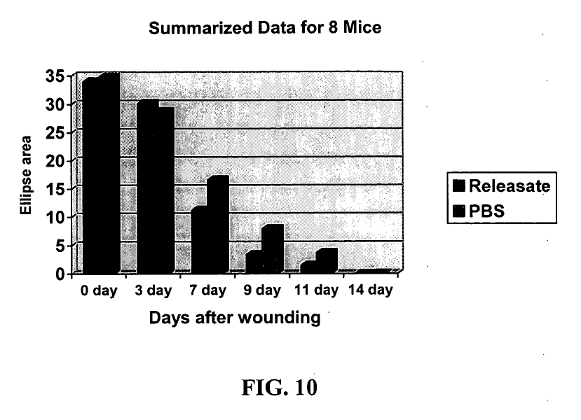

On day 1, three of the test mice removed the bandage and scratched and re-opened only their releasate-treated wound, suggesting that the wound healing process had started in the treated wounds, and caused itching. Furthermore, the treated wounds were visibly redder in color than the control wounds, indicating a more robust vascular response in the treated wounds. The scratching apparently delayed the wound healing on these three mice on day 3. The bandages were re-applied and the mice did not reopen these wounds again. The results are summarized in Table 1 and FIG. 10.

TABLE 1
Effects of growth factors released from ECM on wound healing in mice

| | # of | 0 day | 3 day | 7 day | 9 day | 11 day | 14 day |
|---|---|---|---|---|---|---|---|
| Treatment | mouse | EA | EA | EA | EA | EA | EA |
| ECM | 1 | 30.42 | 20.45 | 13.22 | 6.22 | 3.14 | 0 |
| Releasate | 2 | 32.66 | 30.96 | 7.44 | 1.85 | 0 | 0 |
| | 3 | 27.50 | 20.71 | 4.96 | 1.91 | 1.77 | 0 |
| | 4 | 32.31 | 21.13 | 8.67 | 2.02 | 1.08 | 0 |
| | 5 | 41.80 | 39.36 | 29.59 | 5.10 | 2.28 | 0 |
| | 6 | 33.05 | 38.37 | 12.94 | 7.29 | 1.83 | 0 |
| | 7 | 36.33 | 36.1 | 4.65 | 0.00 | 0.00 | 0 |
| | 8 | 37.58 | 33.85 | 8.17 | 2.95 | 1.80 | 0 |
| Average | | 33.96 ± 1.79 | 30.12 ± 2.24 | 11.21 ± 3.11 | 3.42 ± 0.78 | 1.49 ± 0.39 | 0 |
| PBS | 1 | 38.29 | 29.28 | 23.93 | 18.74 | 11.35 | 0 |
| | 2 | 49.55 | 34.65 | 19.02 | 14.26 | 2.82 | 0 |
| | 3 | 30.61 | 37.96 | 32.71 | 6.83 | 3.97 | 0 |
| | 4 | 39.08 | 23.00 | 9.00 | 4.46 | 1.48 | 0.21 |
| | 5 | 38.64 | 36.48 | 20.83 | 5.08 | 3.98 | 0 |
| | 6 | 26.64 | 18.02 | 7.24 | 0.7 | 0.00 | 0 |
| | 7 | 25.47 | 32.34 | 14.75 | 13.09 | 6.13 | 0 |
| | 8 | 29.71 | 17.95 | 5.13 | 0.35 | 0 | 0 |

| | | | | | | |
|---|---|---|---|---|---|---|
| Average | 34.75 ± 3.01 | 28.71 ± 2.50 | 16.58 ± 3.44 | 7.94 ± 2.25 | 3.72 ± 1.42 | 0.03 ± 0.02 |

As shown in Table 1, on days 7, 9, and 11, the wound closures were significantly better in the releasate-treated wounds than in the PBS-treated wounds. By day 9, one of the releasate-treated wounds (mouse #7) had healed. In addition, on days 9 and 11, the average size of the unclosed areas of the releasate-treated wounds was about half of those of the PBS-treated wounds. By day 14, all wounds treated with releasate had healed, and most were closed between days 11 and 14. Furthermore, hairs in the releasate-treated wounds were thicker. These results indicated that morphogens released from ECM were effective in healing wounds in mice.

### Example 6

### Effects of Temperature on Wound Healing

A cell-free ECM was prepared and treated with a cyclic potential at either 23° C. or 37° C. in the same manner as described above in Examples 2 and 3. Releasates thus prepared at both temperatures were tested for their biological activities as described in Example 3. It was found that the releasate prepared at 37° C. inhibited the growth of cells as described above. In contrast, the releasate prepared at 23° C. did not inhibit cell growth. These results indicated that different temperatures at which the potential was applied to the ECM had different effects on the activity of the releasate.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of generating a morphogen composition from an extracellular matrix, the method comprising:
growing cells on a surface in a fluid under conditions and for a time sufficient to enable the cells to form an extracellular matrix (ECM);
stimulating the extracellular matrix to release morphogens into the fluid; and
collecting the fluid to form a morphogen composition.

2. The method of claim 1, wherein the morphogens are growth factors or differentiating factors.

3. The method of claim 1, wherein the morphogens are differentiating factors, growth factors, bioactive fragments of the ECM, or any combination of two of more of these morphogens.

4. The method of claim 1, wherein the morphogen composition comprises a plurality of morphogens.

5. The method of claim 1, wherein the fluid comprises a biocompatible liquid or biocompatible gel.

6. The method of claim 1, wherein stimulating the extracellular matrix comprises applying an electric potential to the extracellular matrix.

7. The method of claim 6, wherein the electric potential cycles from a negative voltage to a positive voltage and back to a negative voltage.

8. The method of claim 6, wherein the electric potential ranges from −0.3 V to +0.3 V.

9. The method of claim 6, further comprising varying frequency, potential range, potential cycle shape, or potential cycle number of the electric potential to control release and activation of specific morphogens.

10. The method of claim 1, further comprising removing cells from the extracellular matrix to form a cell-free extracellular matrix.

11. A morphogen composition comprising a plurality of morphogens released from a stimulated extracellular matrix.

12. The composition of claim 11, further comprising a biocompatible fluid.

13. The composition of claim 12, wherein the fluid is a buffer.

14. The composition of claim 12, wherein the fluid is a gel.

15. The composition of claim 11 in lyophilized form.

16. The composition of claim 11, wherein the plurality of morphogens comprises any two or more growth factors, differentiating factors, bioactive fragments of the ECM, or any combination of two or more of these morphogens.

17. The composition of claim 16, wherein the plurality of morphogens comprises fibroblast growth factor, transforming growth factor beta, or both.

18. The composition of claim 11, wherein the extracellular matrix is cell-free.

19. The composition of claim 11, wherein the extracellular matrix is stimulated by an electric potential.

20. The composition of claim 19, wherein the electric potential is a negative potential.

21. A method of tissue reconstruction, the method comprising
obtaining an extracellular matrix;
stimulating the extracellular matrix to induce release of morphogens; and
administering the stimulated extracellular matrix to a site where tissue reconstruction is needed.

22. The method of claim 21, further comprising incorporating the stimulated extracellular matrix into a bandage material.

23. The method of claim 21, wherein the site is a tissue defect and the stimulated extracellular matrix includes specific morphogens for treating a specific type of tissue defect.

24. The method of claim 23, wherein the specific type of tissue defect is a laceration, a bum, or a venomous sting.

25. The method of claim 24, wherein the bandage is coded according to the specific type of tissue defect and morphogen that the extracellular matrix of the bandage has released.

26. The method of claim 23, wherein administering the stimulated extracellular matrix comprises placing the bandage in contact with the tissue defect to saturate the tissue defect with morphogens.

27. The method of claim 21, wherein administering the stimulated extracellular matrix comprises placing the stimulated extracellular matrix in contact with a tissue defect in a surgical site to saturate the tissue defect with morphogens.

28. The method of claim 21, wherein the extracellular matrix is cell-free.

29. A method of tissue reconstruction, the method comprising
obtaining a morphogen composition of claim 11; and
administering the morphogen composition to a site where tissue reconstruction is needed.

30. The method of claim 29, wherein the composition comprises a biocompatible liquid.

31. A bandage for application to a tissue defect comprising:
an impermeable membrane forming a sealed cavity;
a first conducting layer arranged within the sealed cavity;
a second conducting layer arranged within the sealed cavity and spaced apart from the first conducting layer;
a buffer reservoir located within the sealed cavity; and
an extracellular matrix arranged within the sealed cavity between the first and second conducting layers and contacting one of the conducting layers.

32. The bandage of claim 31, further comprising a permeable membrane positioned adjacent to the cell-free extracellular matrix and arranged between the first and second conducting layers.

33. The bandage of claim 31, further comprising flexible insulating structural members to maintain separation between the first and second conducting layers during delivery of an electric potential to the cell-free extracellular matrix.

34. The bandage of claim 31, wherein the impermeable membrane comprises an upper impermeable membrane and a lower impermeable membrane sealed together at their respective edges to form the sealed cavity.

35. The bandage of claim 31, wherein the buffer reservoir contains an electrolytic buffer.

36. The bandage of claim 34, wherein the lower impermeable membrane is removable.

37. The bandage of claim 31, wherein the extracellular matrix is cell-free.

38. An electric bandage for application to a tissue defect, the device comprising:
a flexible sheet;
a chamber fixed to the flexible sheet and containing an extracellular matrix;
a first conductor arranged on one side of the chamber;
a second conductor arranged on another side of the chamber;
an electric power source connected to the first and second conductors;
a buffer reservoir arranged to deliver its contents to the extracellular matrix in the chamber; and
a controller connected to the electric power source for applying an electrical potential to the extracellular matrix.

39. The electric bandage of claim 38, wherein the buffer reservoir comprises a liquid impermeable material that can be ruptured by pressure.

40. The electric bandage of claim 38, wherein the buffer reservoir contains an electrolytic buffer.

41. The electric bandage of claim 38, wherein a plurality of morphogens are bound within the extracellular matrix until released by application of electrical potential.

42. The electric bandage of claim 38, wherein the first and second conductors comprises a gold electrode surface, an indium tin oxide electrode surface, or an organic conducting polymer surface.

43. The electric bandage of claim 42, wherein the organic conducting polymer surface is electrochemically grown or deposited on a metal or non-metallic substrate.

44. The electric bandage of claim 38, wherein the controller applies an electric potential in a range of −0.3 V to +0.3 V.

45. The electric bandage of claim 38, wherein the extracellular matrix is cell-free and includes specific morphogens for treating a specific type of tissue defect.

46. A pharmaceutical composition comprising a pharmaceutically acceptable carrier and a morphogen composition of claim 11.
